(19) United States
(12) Patent Application Publication
(10) Pub. No.: US 2011/0301427 A1
(43) Pub. Date: Dec. 8, 2011

(54) Acoustic physiological monitoring device and large noise handling method for use thereon

(76) Inventors: Yongji Fu, Vancouver, WA (US); Bryan Severt Hallberg, Vancouver, WA (US)

(21) Appl. No.: 12/802,332

(22) Filed: Jun. 4, 2010

(51) Int. Cl. A61B 5/00

(52) U.S. Cl. 600/300

(57) ABSTRACT

A physiological monitoring device and large noise handling method for use on such a device in which a reliable estimate of a physiological parameter is ensured by identifying and replacing large noise components of a physiological signal prior to estimation. An estimation period for a physiological parameter is segmented into time windows. Noisy time windows within the estimation period are identified. The noisy time windows are replaced with replacement time windows having a baseline amplitude. An estimate of the physiological parameter for the estimation period is calculated using the replacement time windows in lieu of the noisy time windows, and is outputted. If the share of noisy time windows exceeds a predetermined limit share, calculating and/or outputting of an estimate may be precluded. The physiological parameter may be heart rate.

### BACKGROUND OF THE INVENTION

The present invention relates to physiological monitoring and, more particularly, to large noise handling on an acoustic physiological monitoring device.

Real-time monitoring of the physiological state of people who suffer from chronic diseases is an important aspect of chronic disease management. Real-time physiological monitoring is in widespread use in managing cardiovascular, pulmonary and respiratory disease, and also widely used in other contexts, such as elder care. Some real-time physiological monitoring devices monitor the physiological state of human subjects by detecting and evaluating acoustic signals that contain body sounds.

One problem encountered in real-time acoustic physiological monitoring is parameter estimation error caused by large noise. Real-time acoustic physiological monitoring is often performed using a portable (e.g. wearable) device that continually acquires and analyzes an acoustic physiological signal, such as a signal that includes heart and lung sounds, as a person wearing the device goes about his or her daily life. The acquired physiological signal can be temporarily affected by large noise, such as speech or environmental noise. This can result in erroneous estimation of physiological parameters by the device and outputting of erroneous estimates. Reliance on these erroneous estimates can have serious adverse consequences on the health of the person being monitored. For example, erroneous estimates can lead the person or his or her clinician to improperly interpret physiological state and cause the person to undergo treatment that is not medically indicated, or forego treatment that is medically indicated.

One way to prevent reliance on erroneous estimates is to unconditionally reject estimates of physiological parameters generated in the presence of large noise. However, unconditional rejection can present difficulties. For example, to generate a reliable estimate of certain physiological parameters, such as heart rate, a physiological signal must be evaluated over a sustained estimation period (e.g. 15 seconds). If large noise is present in the physiological signal for a short time within the estimation period and a rule of unconditional rejection is enforced, no estimate will be available for the entire estimation period and the valuable real-time data provisioning feature of acoustic physiological monitoring will be compromised.

### SUMMARY OF THE INVENTION

The present invention provides a physiological monitoring device and large noise handling method for use on such a device in which a reliable estimate of a physiological parameter is ensured by identifying and replacing large noise components of a physiological signal prior to estimation. An estimation period for a physiological parameter is segmented into time windows. Noisy time windows within the estimation period are identified. The noisy time windows are replaced with replacement time windows having a baseline amplitude. An estimate of the physiological parameter for the estimation period is calculated using the replacement time windows in lieu of the noisy time windows, and is outputted. If the share of noisy time windows exceeds a predetermined limit share, calculating and/or outputting of an estimate may be precluded. The physiological parameter may be heart rate.

In one aspect of the invention, therefore, a physiological monitoring device comprises an acoustic transducer; a processor communicatively coupled with the acoustic transducer; and an output interface communicatively coupled with the processor, wherein a physiological signal detected by the acoustic transducer is transmitted to the processor, and wherein under control of the processor the device segments the physiological signal, in an estimation period for a physiological parameter, into initial time windows, identifies one or more noisy time windows among the initial time windows, replaces the noisy time windows with replacement time windows having a baseline amplitude, calculates an estimate of the physiological parameter using amplitudes of the physiological signal in non-replaced initial time windows and the replacement time windows, and transmits the estimate to the output interface whereon the estimate is outputted.

In some embodiments, under control of the processor the device determines the baseline amplitude using average amplitudes of the physiological signal in a subset of the initial time windows having the lowest average amplitudes.

In some embodiments, under control of the processor the device identifies the noisy time windows based on comparisons involving average amplitudes of the physiological signal in one or more of the initial time windows and the baseline amplitude.

In some embodiments, under control of the processor the device compares a share of the noisy time windows with a predetermined limit share and conditions outputting of the estimate on a determination that the share of the noisy time windows does not exceed the predetermined limit share.

In some embodiments, under control of the processor the device applies a band-pass filter to the physiological signal.

In some embodiments, under control of the processor the device calculates the estimate at least in part by analyzing a peak amplitude of an autocorrelation result obtained by applying an autocorrelation function to amplitudes of the physiological signal in the non-replaced initial time windows and the replacement time windows.

In some embodiments, the physiological parameter is a heart rate.

In some embodiments, the estimate is displayed on a display screen of the output interface.

In some embodiments, the device is portable.

In another aspect of the invention, a large noise handling method for a physiological monitoring device comprises the steps of detecting by the device a physiological signal; segmenting by the device the physiological signal, in an estimation period for a physiological parameter, into initial time windows; identifying by the device one or more noisy time windows among the initial time windows; replacing by the device the noisy time windows with replacement time windows having a baseline amplitude; calculating by the device an estimate of the physiological parameter using amplitudes of the physiological signal in non-replaced initial time windows and the replacement time windows; and outputting by the device the estimate.

These and other aspects of the invention will be better understood by reference to the following detailed description taken in conjunction with the drawings that are briefly described below. Of course, the invention is defined by the appended claims.

### BRIEF DESCRIPTION OF THE DRAWINGS

FIG. 1 shows a physiological monitoring device in some embodiments of the invention.

FIG. 2 shows a large noise handling method for a physiological monitoring device in some embodiments of the invention.

FIG. 3 is a plot illustrating how a baseline amplitude is determined in some embodiments of the invention.

FIG. 4 is a plot illustrating how noisy time windows are replaced with replacement time windows having the baseline amplitude in some embodiments of the invention.

FIG. 5 is a plot illustrating how a physiological parameter is calculated in some embodiments of the invention.

### DETAILED DESCRIPTION OF A PREFERRED EMBODIMENT

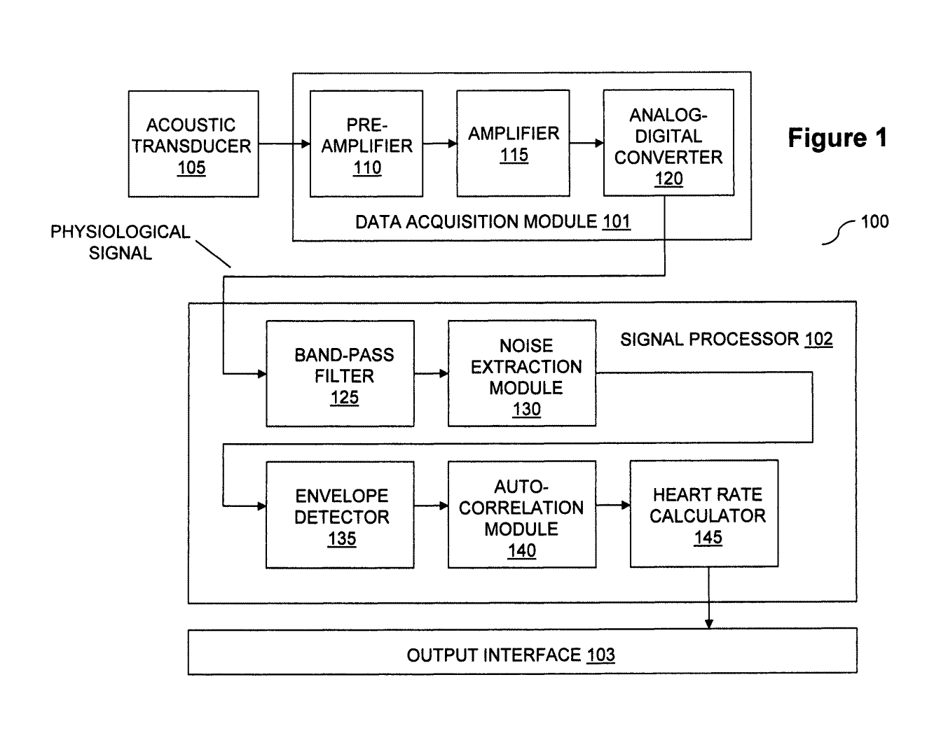

FIG. 1 shows a physiological monitoring device 100 in some embodiments of the invention. Device 100 has an acoustic transducer 105 which during operation is positioned on the body of the human subject being monitored, such as on the person's trachea, chest or back. Transducer 105 is communicatively coupled with data acquisition module 101 that includes a pre-amplifier 110, amplifier 115 and an analog-to-digital (A/D) converter 120. A/D converter 120 continually transmits an acoustic physiological signal detected by transducer 105, as modified by amplifiers 110, 115, to a signal processor 102. Using the physiological signal, signal processor 102 generates heart rate estimates for heart rate estimation periods and transmits the heart rate estimates to an output interface 103, which may display the heart rate estimates on a display screen. In some embodiments, device 100 is a portable ambulatory monitoring device that may be attached to the subject's clothing (e.g. clipped-on) or carried by the subject (e.g. hand-held).

Transducer 105 detects sound at a position on the subject's body, such as the trachea, chest or back. Transducer 105 in some embodiments comprises an omni-directional microphone housed in an air chamber. Transducer 105 outputs to data acquisition module 101 as an analog voltage a raw physiological signal based on detected sound.

At data acquisition module 101, pre-amplifier 110 provides impedance match for the raw physiological signal received from transducer 105 and amplifies the raw physiological signal. Amplifier 115 further amplifies the raw physiological signal received from amplifier 110 to the range of +/−1 V. A/D converter 120 performs A/D conversion on the raw physiological signal received from amplifier 115 and transmits the raw physiological signal to signal processor 102 for analysis.

At signal processor 102, the raw physiological signal is processed to generate and transmit to output interface 103 continual heart rate estimates. In some embodiments, signal processor 102 is a microprocessor having software executable thereon for performing signal processing on the raw physiological signal received from data acquisition module 101. In other embodiments, all or part of the functions of signal processor 102 may be performed in custom logic, such as one or more application specific integrated circuits (ASIC).

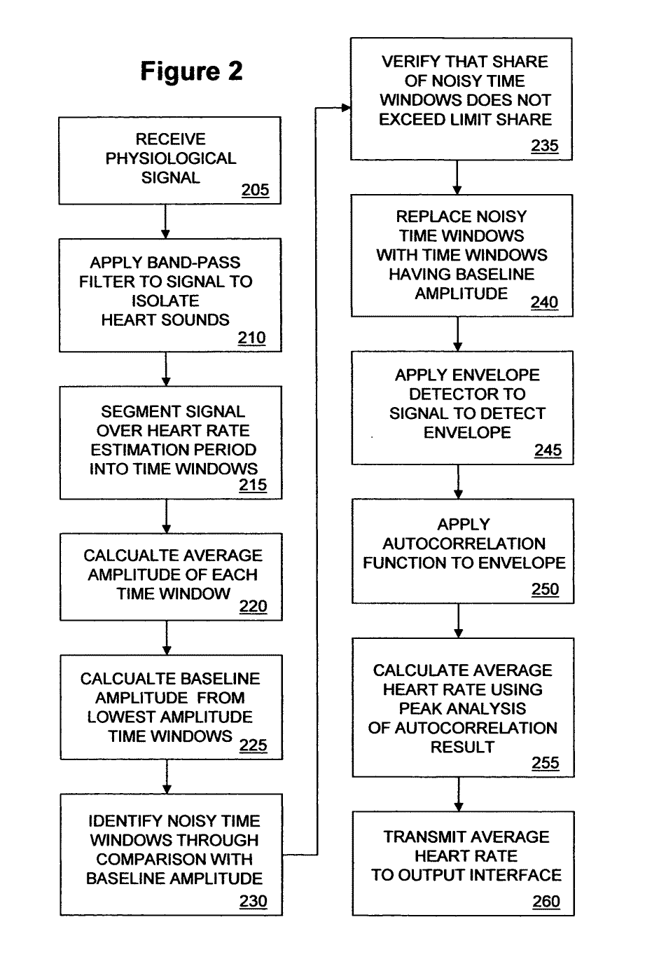

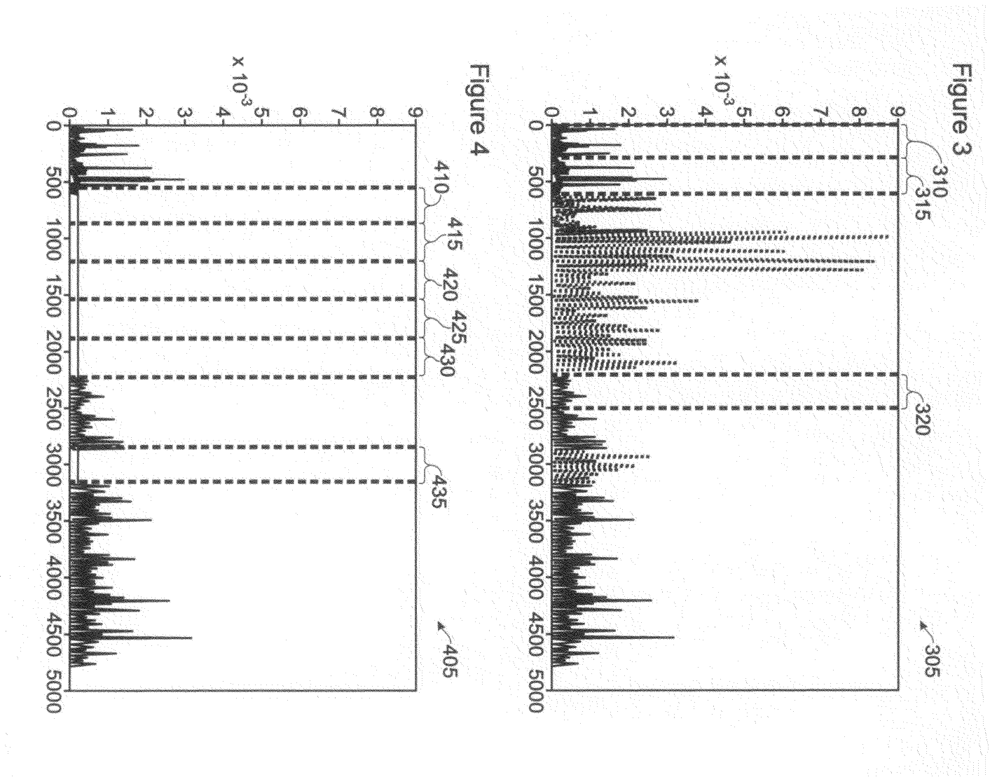

Signal processor 102 includes a band-pass filter 125, a noise extraction module 130, an envelope detector 135, an autocorrelation module 140 and a heart rate calculator 145. Steps of large noise handling method performed by signal processor 102 to generate heart rate estimates in some embodiments of the invention are shown in FIG. 2 and will be described by reference to FIGS. 3-5.

Initially, the raw physiological signal is received (205) from data acquisition module 101. The raw physiological signal is noisy and heart sounds are intermingled with other body sounds, such as lung sounds, as well as signal noise originating from the background environment, motion and/or speech.

Next, band-pass filter 125 filters the physiological signal to isolate heart sounds (210), in particular, a pulse sequence. In some embodiments, band-pass filter 125 is a fifth order Butterworth filter having cutoff frequencies at 20 and 120 Hz.

Next, noise extraction module 130 segments the physiological detected over a heart rate estimation period into initial time windows (215). For example, device 100 may be configured to generate four heart rate estimates per minute, such that the operative heart rate estimation period is 15 seconds. Continuing with the example, noise extraction module 135 segments the 15-second heart rate estimation period into 15 one-second initial time windows for analysis.

Next, noise extraction module 130 calculates an average signal amplitude for each initial time window (220). Continuing with the above example, noise extraction module 135 calculates a mean signal amplitude for each of the 15 one-second initial time windows.

Next, noise extraction module 130 calculates a baseline signal amplitude for the heart rate estimation period from the lowest amplitude initial time windows (225). Continuing with the above example, noise extraction module 130 identifies among the 15 one-second initial time windows in the heart rate estimation period the three initial time windows that have the three lowest mean signal amplitudes, respectively. Noise extraction module 130 then calculates a baseline amplitude as the mean of the three lowest mean signal amplitudes. FIG. 3 is a plot 305 that illustrates how a baseline amplitude is determined in some embodiments of the invention. Plot 305 shows a physiological signal that varies widely in amplitude over a heart estimation period. The baseline amplitude is calculated as the mean of three initial time windows 310, 315, 320 within the heart rate estimation period that have the lowest mean signal amplitudes.

Next, noise extraction module 130 identifies noisy time windows among the initial time windows through comparison with the baseline signal amplitude (230). Continuing with the above example, noise extraction module 130 identifies from the 15 one-second initial time windows in the heart rate estimation period all initial time windows whose mean signal amplitude is more than twice the baseline amplitude, and classifies those initial time windows as noisy time windows.

Next, noise extraction module 130 verifies that the share of the initial time windows that have been classified as noisy time windows does not exceed a predetermined limit share (235). Continuing with the above example, noise extraction module 130 determines whether more than half (e.g. eight out of 15) of the initial time windows have been classified as noisy time windows. If so, the good (i.e. non-noisy) share of the physiological signal in the heart rate estimation period is deemed too small to form the basis of a reliable heart rate estimate and the attempt to generate a heart rate estimate for the heart rate estimation period is aborted. If not, the good share of the physiological signal in the heart rate estimation period is deemed large enough to form the basis of a reliable heart rate estimate and the flow proceeds to Step 240.

Next, noise extraction module 130 replaces noisy time windows with replacement time windows having the baseline signal amplitude across the entire time window (240). Continuing with the above example, FIG. 4 is a plot 405 that illustrates how noisy time windows from FIG. 3 are replaced with replacement time windows having the baseline amplitude in some embodiments of the invention. In plot 405, the six initial time windows from FIG. 3 identified as having mean signal amplitudes of more than twice the baseline amplitude are shown to have been replaced with the replacement time windows 410, 415, 420, 425, 430, 435 having the baseline amplitude.

Next, envelope detector 135 is applied to the physiological signal to detect a signal envelope (245). The signal envelope may be detected using a standard deviation method or an entropy method, for example, that identifies and extracts the relatively slowly changing periodic components of the physiological signal.

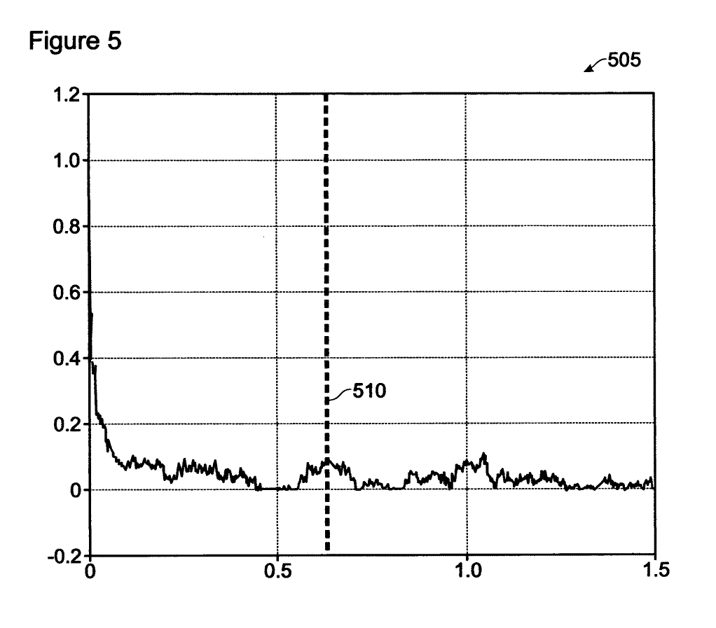

Next, autocorrelation module 140 is applied to the detected envelope to generate an autocorrelation result that identifies the fundamental periodicity in the physiological signal (250). Continuing with the above example, FIG. 5 is a plot 505 illustrating an autocorrelation result from which heart rate is estimated. The autocorrelation result exhibits a maximum peak at zero time delay and lesser peaks at positive time delays. The center of the highest peak between 0.33 and 1.50 seconds corresponds to the average pulse period. The range of 0.33 and 1.50 seconds is selected for peak detection because a pulse period of 0.33 and 1.50 seconds corresponds to a heart rate of between 40 and 182 beats per minute that may be experienced by human subjects.

Next, heart rate calculator 145 determines an average pulse period using peak analysis of the autocorrelation result (255). The average pulse period is identified as the peak-to-peak time difference between the maximum peak at zero time delay and the highest peak between 0.33 and 1.50 seconds. In the example shown in FIG. 5, highest peak 510 between 0.33 and 1.50 seconds is centered at 0.68 seconds, which is identified as the average pulse period. Heart rate calculator 145 estimates heart rate using the average pulse period. More particularly, a heart rate estimate in beats per minute is calculated as 60 divided by the average pulse period. Returning to the example shown in FIG. 5, the heart rate is estimated to be 60/0.68, or 88.2 beats per minute.

Finally, signal processor 102 transmits the heart rate estimate to output interface 103 (260) for display and/or further processing. Output interface 103 includes a user interface having a liquid crystal display or light emitting diode screen that displays the heart rate estimate to the subject being monitored. Output interface 103 may additionally have a data management interface to an internal or external data management system that stores the heart rate estimate and/or a network interface that transmits the heart rate estimate to a remote monitoring device, such as a monitoring device at a clinician facility.

Signal processor 102 re-performs the above steps to generate and output heart rate estimates for subsequent heart rate estimation periods. In some embodiments, consecutive heart rate estimation periods are contiguous.

The numerical values discussed and applied in the above steps are merely representative. By way of example, a physiological monitoring device operating within the scope of the present invention may use a longer or shorter estimation period, may segment the estimation period into a larger or smaller number of initial time windows, may calculate the baseline amplitude using a larger or smaller number of initial time windows, may identify initial time windows as noisy through comparison with a larger or smaller multiple of the baseline amplitude, and/or may require a larger or smaller share of the physiological signal to be good in order to proceed with physiological parameter estimation. Moreover, the present invention may be applied to facilitate estimation of physiological parameters other than heart rate, such as respiratory parameters.

Accordingly, it will be appreciated by those of ordinary skill in the art that the invention can be embodied in other specific forms without departing from the spirit or essential character hereof. The present description is considered in all respects to be illustrative and not restrictive. The scope of the invention is indicated by the appended claims, and all changes that come with in the meaning and range of equivalents thereof are intended to be embraced therein.

What is claimed is:

1. A physiological monitoring device, comprising:
an acoustic transducer;
a processor communicatively coupled with the acoustic transducer; and
an output interface communicatively coupled with the processor, wherein a physiological signal detected by the acoustic transducer is transmitted to the processor, and wherein under control of the processor the device segments the physiological signal, in an estimation period for a physiological parameter, into initial time windows, identifies one or more noisy time windows among the initial time windows, replaces the noisy time windows with replacement time windows having a baseline amplitude, calculates an estimate of the physiological parameter using amplitudes of the physiological signal in non-replaced initial time windows and the replacement time windows, and transmits the estimate to the output interface whereon the estimate is outputted.

2. The device of claim 1, wherein under control of the processor the device determines the baseline amplitude using average amplitudes of the physiological signal in a subset of the initial time windows having the lowest average amplitudes.

3. The device of claim 1, wherein under control of the processor the device identifies the noisy time windows based on comparisons involving average amplitudes of the physiological signal in one or more of the initial time windows and the baseline amplitude.

4. The device of claim 1, wherein under control of the processor the device compares a share of the noisy time windows with a predetermined limit share and conditions outputting of the estimate on a determination that the share of the noisy time windows does not exceed the predetermined limit share.

5. The device of claim 1, wherein under control of the processor the device applies a band-pass filter to the physiological signal.

6. The device of claim 1, wherein under control of the processor the device calculates the estimate at least in part by analyzing a peak amplitude of an autocorrelation result obtained by applying an autocorrelation function to amplitudes of the physiological signal in the non-replaced initial time windows and the replacement time windows.

7. The device of claim 1, wherein the physiological parameter is a heart rate.

8. The device of claim 1, wherein the estimate is displayed on a display screen of the output interface.

9. The device of claim 1, wherein the device is portable.

10. A large noise handling method for a physiological monitoring device, comprising the steps of:
detecting by the device a physiological signal;
segmenting by the device the physiological signal, in an estimation period for a physiological parameter, into initial time windows;
identifying by the device one or more noisy time windows among the initial time windows;
replacing by the device the noisy time windows with replacement time windows having a baseline amplitude;
calculating by the device an estimate of the physiological parameter using amplitudes of the physiological signal in non-replaced initial time windows and the replacement time windows; and
outputting by the device the estimate.

11. The method of claim 10, further comprising the step of determining by the device the baseline amplitude using average amplitudes of the physiological signal in a subset of the initial time windows having the lowest average amplitudes.

12. The method of claim 10, wherein the device identifies the noisy time windows based on comparisons involving average amplitudes of the physiological signal in one or more of the initial time windows and the baseline amplitude.

13. The method of claim 10, further comprising the steps of comparing by the device a share of the noisy time windows with a predetermined limit share and conditioning by the device outputting of the estimate on a determination that the share of the noisy time windows does not exceed the predetermined limit share.

14. The method of claim 10, further comprising the step of applying by the device a band-pass filter to the physiological signal.

15. The method of claim 10, wherein the device calculates the estimate at least in part by analyzing a peak amplitude of an autocorrelation result obtained by applying an autocorrelation function to amplitudes of the physiological signal in the non-replaced initial time windows and the replacement time windows.

16. The method of claim 10, wherein the physiological parameter is a heart rate.

17. The method of claim 10, wherein the estimate is displayed on a display screen of the output interface.

18. The method of claim 10, wherein the device is portable.
